(19) United States
(12) Patent Application Publication
(10) Pub. No.: US 2002/0172972 A1
(43) Pub. Date: Nov. 21, 2002

(54) Use of a selectively inactivatable enzyme to digest contaminating nucleic acid

(75) Inventors: Stanley Tabor, Brookline, MA (US); Charles C. Richardson, Newton, MA (US)

Correspondence Address: BANNER & WITCOFF, LTD., 28 STATE STREET, 28th FLOOR, BOSTON, MA, 02109, US

(73) Assignee: President and Fellows of Harvard College, Cambridge, MA

(21) Appl. No.: 10/146,872

(22) Filed: May 15, 2002

Related U.S. Application Data
(60) Provisional application No. 60/290,890, filed on May 15, 2001.

(51) Int. Cl. C12Q 1/68; C12P 19/34

(52) U.S. Cl. 435/6; 435/91.2

(57) ABSTRACT

Methods and kits for rendering inert contaminating nucleic acids are provided. The methods and kits include an enzyme capable of digesting the contaminating nucleic acids, an activator which activates the enzyme, and an inactivating agent capable of binding to or displacing the activator from the enzyme thus rendering the enzyme inactive.

### RELATED U.S. APPLICATION(S)

This application claims the benefit of the filing date of U.S. Provisional Application Serial No. 60/290,890, filed May 15, 2001, hereby incorporated by reference in its entirety.

### STATEMENT OF GOVERNMENT INTERESTS

This invention was made with Government support under Grant Number DE-FG02-96ER62251 awarded by the Department of Energy and Grant Number GM54397-36 awarded by the National Institutes of Health. The Government has certain rights in the invention.

### FIELD OF INVENTION

This application relates to methods, reagents and kits for the removal of contaminating nucleic acids from reagent solutions used in, for example, PCR, forensics, and the like. More particularly, the method disclosed here provides for removal of contaminating nucleic acids from reagent solutions to provide for increased efficiency of DNA amplification.

### BACKGROUND OF THE INVENTION

Reagents commonly used to amplify nucleic acids may contain unwanted nucleic acid contaminants that could drastically interfere with standard amplification protocols and procedures, such as polymerase chain reaction (PCR). Although the enzymes and reagents used for PCR have undergone purification using standard protein purification techniques, there exists a high probability that DNA contaminants may be found in one or more of the reagent solutions.

To avoid the presence of contaminating DNA, several methods are commonly used. These methods include mechanical methods such as the use of sterile hoods and pipette tips that contain filters. The mechanical methods are undesirable because sterilization of pipette tips, and other equipment used for PCR, is time consuming and expensive. Additionally, one must purchase an expensive sterile hood, such as a laminar flow hood, and have the space to permanently set up the hood. Other methods include enzymatic methods such as the use of uracil glycosylase to specifically degrade PCR products. See Longo et al., Gene 93, 125 (1990). One disadvantage of this process, however, is that it only removes contaminating DNA from previous PCR reactions that contain uracil in their DNA. There, exists a need in the art for a rapid method of removing contaminating DNA from reagents without the requirement of further purification techniques.

Accordingly, it is an object of the present invention to provide methods of digesting contaminating nucleic acids in a reaction mixture. Certain preferred embodiments of the present invention are especially useful to increase amplification of target nucleic acids by digesting contaminating nucleic acids in a reaction mixture without requiring purification of the solutions prior to performing DNA synthesis reactions.

### SUMMARY

Embodiments of the present invention are directed to the use of enzymes, e.g. selectively activatable enzymes, and accordingly, selectively inactivatable enzymes, to remove, digest, degrade or otherwise inactivate nucleic acids, e.g. contaminating nucleic acids, which may be present in a reagent or a solution, e.g. a solution used in a reaction mixture. Typically, the nucleic acids will be considered contaminants, however, the invention is generally applicable to all nucleic acids that are desired to be removed, digested, degraded or otherwise inactivated. The terms “selectively activated” and “selectively inactivated” refer to the nucleic acid digesting ability of the enzyme to be started and stopped or initiated and terminated using, for example, cations and chelating agents. According to the methods of the present invention, the nucleic acids are altered by digestion or degradation in a manner to prevent their further activity or reactivity in nucleic acid amplification methods, i.e. the nucleic acids have become inactivated or they have been removed from further reactivity. According to this embodiment of the present invention, purification steps of prior art methods to remove nucleic acids or fragments thereof are avoided.

In accordance with a first aspect, a method of rendering contaminating nucleic acid inert is provided. An enzyme is added to a solution containing the nucleic acid. According to one embodiment, the enzyme has maximal activity in the presence of bound activator and minimal activity in the absence of bound activator. The enzyme is capable of rendering contaminating nucleic acid inert in the presence of bound activator and incapable of rendering contaminating nucleic acid inert in the absence of bound activator. The enzyme having bound activator is incubated for a period of time sufficient to allow the enzyme to render contaminating nucleic acid inert. Bound activator is then removed from the enzyme to inhibit the enzyme from further acting on contaminating or other nucleic acid.

In accordance with a second aspect, a method of digesting contaminating nucleic acid is provided. An enzyme is added to a solution for performing a DNA synthesis reaction. The enzyme is capable of digesting the contaminating nucleic acid in a first state and incapable of digesting the contaminating nucleic acid in a second state. The enzyme is converted into the first state by adding an activator. The enzyme in the first state is incubated in a manner to allow for digestion of the contaminating nucleic acid by the enzyme in the first state. The enzyme is converted to the second state by adding an inactivating agent.

In accordance with a third aspect, a method of digesting contaminating nucleic acid is further provided. An enzyme is added to a solution for performing a DNA synthesis reaction. The enzyme is capable of digesting the contaminating nucleic acid in a first state and incapable of digesting the contaminating nucleic acid in a second state. The enzyme is converted into the first state by adding an activator. The enzyme in the first state is then incubated in a manner to allow for digestion of the contaminating nucleic acid by the enzyme. The enzyme is converted to the second state by removing the activator.

In accordance with another aspect, a method of amplifying nucleic acids is provided using selectively activatable and/or selectively inactivatable enzymes to digest nucleic acids in a reaction mixture which is later then used in amplification methods such as PCR or isothermal amplification systems. For example, in one aspect a method of digesting contaminating nucleic acid is provided. A selectively inactivatable enzyme is added to a solution for performing a DNA synthesis reaction, in which the solution comprises contaminating nucleic acid. An activator is added to the solution comprising the selectively inactivatable enzyme. The activator binds to the selectively inactivatable enzyme to form a selectively activatable enzyme. The solution comprising the selectively activatable enzyme is incubated in a manner to allow for digestion of the contaminating nucleic acid by the selectively activatable enzyme. The selectively activatable enzyme is converted into the selectively inactivatable enzyme by removing the activator from the selectively activatable enzyme. The solution is then used in an amplification method.

In accordance with an additional aspect, a method is provided of digesting nucleic acids in a reaction mixture whereby a selectively activatable and/or selectively inactivatable enzyme, e.g. a nuclease, is allowed to degrade or digest the nucleic acids in a manner to produce individual nucleotides or oligonucleotides which lack a 3′ hydroxyl group.

In accordance with an additional aspect, a method is provided in which nucleic acids are altered by digestion or degradation in a manner to prevent their further reactivity in nucleic acid amplification methods, i.e. the nucleic acids have become inactivated or they have been removed from further reactivity. It is a significant advantage that no purification steps are required prior to performing subsequent assays and methods, such as PCR or isothermal DNA amplification methods, for example. That is, purification steps of prior art methods to remove nucleic acids or fragments thereof are avoided using the methods, kits and assays disclosed here.

In accordance with another aspect, a method of digesting contaminating nucleic acids is provided. The method comprises adding an effective amount of an enzyme, capable of digesting nucleic acids and capable of being selectively activatable and inactivatable, to a reagent or a reagent mixture, activating the enzyme by adding one or more activators, providing a suitable time for the activatable enzyme to digest any contaminating nucleic acid present in the reagent or reagent mixture, and inactivating the enzyme, for example, by removing any bound activator, by binding with an inhibitor of the enzyme and/or otherwise degrading the enzyme to prevent further nucleic acid digesting activity of the enzyme. Suitable activators generally depend on the nature and characteristics of the selectively activatable enzyme, and those skilled in the art given the benefit of this disclosure will be able to select suitable activators for a given selectively activatable enzyme. For example, methods for the selection of DNA aptamers that bind to and inhibit Taq DNA polymerase include Lin, Y., Jayasena S. D., J. Mol. Biol. Aug. 8, 1997:271(1): 100-111, the entire disclosure of which is incorporated herein by reference for all purposes, the teachings of which could be used to select for DNA or RNA aptamers that bind to and inhibit micrococcal nuclease or other enzymes useful in embodiments of the present invention. Similarly, methods for the use of an antibody to bind to and inhibit Taq DNA polymerase include Kellog, D. E., Rybalkin, I., Chen, S., Mukhamedova, N., Vlasik, T., Siebert, P. D., and Chenchik, A., Biotechniques June 1994; 16(6): 1134-7, the entire disclosure of each of which is incorporated herein by reference for all purposes, the teachings of which could be used to select for DNA or RNA antibodies that bind to and inhibit micrococcal nuclease or other enzymes useful in embodiments of the present invention. Preferably, the use of certain aptamers and antibodies as inhibitors are applicable in isothermal amplification techniques to minimize any dissociation which may result from high temperatures.

In accordance with another aspect, a method of digesting contaminating nucleic acid in a DNA synthesis reaction comprising the steps of adding a selectively inactivatable enzyme to a solution comprising contaminating nucleic acid, adding an activator to the solution comprising the selectively inactivatable enzyme to activate the selectively inactivatable enzyme, incubating the solution to allow for digestion of the contaminating nucleic acid, inactivating the selectively inactivatable enzyme, and adding a DNA template to the solution for amplification of the DNA template is provided.

In accordance with an additional aspect, a method of amplifying DNA comprising (a) treating one or more reagent solutions with an enzyme in a manner to digest contaminating nucleic acids, wherein the reagent solutions comprise at least a DNA polymerase, a buffer, free nucleotides and one or more salt solutions; (b) inactivating the enzyme; (c) adding a DNA template and optionally nucleotide primers to the solution; (d) amplifying the DNA template by using a process comprising the steps of:

(i) optionally denaturing double-stranded DNA template if present;

(ii) allowing the primers to anneal to DNA template and allowing binding of the polymerase to the annealed DNA/primer complex; and

(iii) elongating the DNA strands using the free nucleotides and the polymerase; and (e) repeating step (d) for a predetermined number of cycles to amplify the DNA template is disclosed.

In accordance with another aspect, a kit comprising a selectively activated enzyme capable of digesting contaminating nucleic acids in a manner to produce nucleotides having a 3′-phosphate group and a 5′-hydroxyl group, an activator capable of binding to the enzyme and activating the enzyme, and an inactivating agent that binds to the activator is provided.

In accordance with an additional aspect, a kit comprising a selectively activated enzyme capable of digesting contaminating nucleic acids in a manner to produce nucleotides having a 3′-phosphate group and a 5′-hydroxyl group, an activator capable of binding to the enzyme and activating the enzyme, and a metal species capable of displacing the bound activator, wherein the metal species binds to a same site as the activator with a higher binding affinity than the activator is disclosed.

In accordance with another aspect, a kit comprising a selectively activatable enzyme capable of digesting contaminating nucleic acids in a manner to produce nucleotides or oligonucleotides having a 3′-phosphate group and a 5′-hydroxyl group, an activator capable of binding to the enzyme and activating the enzyme, a metal species capable of displacing the bound activator, wherein the metal species binds to a same site as the activator with a higher binding affinity than the activator, and an inactivating agent that binds to the activator is provided.

In accordance with another aspect, a kit for performing PCR comprising a selectively activatable enzyme, an selectively activatable enzyme activator, an inactivating agent which renders the activatable enzyme inactive, at least one DNA polymerase, free nucleotides (e.g. dNTPs such as dATP, dGTP, dCTP and dTTP), and suitable buffer solutions is provided.

It will be recognized by those skilled in the art that the methods, assays and kits disclosed here represent a significant technological advance. The methods provide rapid decontamination of reagents and solutions without the need to purify the reagents prior to performing any assays, such as PCR or isothermal amplification methods. These and other objects, features and advantages of the present invention will become apparent from the following description taken in conjunction with the accompanying drawings.

### BRIEF DESCRIPTION OF DRAWINGS

Certain preferred embodiments of the invention will be described below with reference to the attached figures in which:

FIG. 1 is a diagram of a process for removal of contaminating nucleic acids in accordance with certain preferred embodiments;

FIG. 2 is a diagram of a process for amplifying DNA in accordance with certain preferred embodiments;

FIG. 3 is a diagram of a process for amplifying DNA in accordance with certain preferred embodiments; and

FIG. 4 is a diagram of a process for heat inactivation of an enzyme in accordance with certain preferred embodiments.

### DETAILED DESCRIPTION OF CERTAIN PREFERRED EMBODIMENTS

Certain preferred embodiments of the present invention are directed to methods, reagents and kits useful for inactivating nucleic acids or otherwise rendering them inert to, for example, DNA or RNA amplification reactions, as desired in certain applications. The precise use, including the choice of variables such as concentrations, volumes, incubation times, incubation temperatures, and the like will depend in large part on the particular application for which it is intended. It is to be understood that one of skill in the art will be able to identify suitable variables based on the present disclosure. For convenience in this more detailed description of certain preferred embodiments, the method disclosed here will generally be of a type suitable for use in removal of contaminating DNA in reagents and solutions, such as those reagents and solutions suitable for use in PCR or in isothermal DNA amplification methods. It will be within the ability of those skilled in the art, however, given the benefit of this disclosure, to select and optimize suitable conditions for using the methods in accordance with the principles of the present invention, suitable for these and other types of applications.

The following terms are intended to have the following meanings unless otherwise clear from the context of the usage of the term.

As used here “enzyme capable of rendering contaminating nucleic acid inert” refers to enzymes which can modify nucleic acids such that the nucleic acids cannot substantially interfere with subsequent chemical analysis and/or procedures, such as PCR or isothermal DNA amplification, for example. Preferably, the enzymes render the contaminating nucleic acid inert by digesting the contaminating nucleic acid to provide products which cannot react with the other components in the solutions. For example, in certain embodiments, the enzyme renders the nucleic acids inert by degrading the contaminating nucleic acid to yield phosphate moieties at the 3′ ends and hydroxyl moieties at the 5′ ends of nucleotides or nucleotide fragments. Such nucleotides or nucleotide fragments are incapable of altering or otherwise interfering with amplification of a DNA template, and, therefore, the digestion products do not need to be removed prior to amplification of the DNA template.

As used here “digesting” refers to breaking bonds, e.g. phosphodiester bonds, between two or more chemical groups. Preferably digesting refers to breaking bonds between two or more nucleotides, such that the free nucleotides and nucleotide fragments are produced. More preferably, digesting refers to breaking bonds between two or more nucleotides such that the products are rendered inert (as discussed below).

As used here “contaminating nucleic acid” refers to nucleic acid which is present in a solution, reagent, apparatus, etc., but undesirable. That is, the contaminating nucleic acid is any nucleic acid which is not intended to by amplified or further characterized. For example, nucleic acid which is present in a reagent or solution for performing a DNA synthesis reaction, prior to adding the DNA template to be amplified, is considered contaminating nucleic acid because it is undesirable to amplify or to characterize the contaminating DNA. It is desirable to remove such contaminating nucleic acid such that when the DNA template to be amplified is added to the solution, the contaminating nucleic acid will not interfere with the DNA synthesis reaction.

As used here “enzyme” refers to biological catalysts which typically are proteins but may in certain instances have nucleic acid constituents, e.g. ribozymes. The enzyme preferably has a high turnover rate and/or high catalytic power such that digestion of the contaminating nucleic acid occurs in a rapid manner. Preferably, the activity of the enzyme is high enough to provide at least about 90% degradation of contaminating nucleic acid during the incubation period, more preferably at least about 95% digestion and most preferably to provide at least about 99% digestion, e.g. at least about 99.9% digestion, by the end of the incubation period.

As used here “a solution for performing a DNA synthesis reaction” refers to any or all solutions which are typically used to perform DNA synthesis reactions, such as PCR and/or isothermal DNA amplification methods. The solution typically comprises buffer, salts, free nucleotides, and optionally primers and a DNA template to be amplified.

As used here “reaction mixture” refers to the combination of reagents or solutions which is used to carry out one or more chemical analyses or assays. Preferably, the reaction mixture includes all necessary components to carry out an assay, such as polymerase chain reaction, for example.

As used here “first state” of an enzyme refers to the state in which the enzyme is active. That is, the enzyme has maximal activity when it is in the first state such that digestion of contaminating nucleic acid can occur efficiently and rapidly.

As used here “second state” of an enzyme refers to the state in which the enzyme is substantially minimal or no activity. That is, when the enzyme is in the second state substantially no digestion of nucleic acid occurs, e.g. <1% digestion occurs. The enzyme typically is converted from the first state into the second state, or vice versa, by using an activator and/or an inactivating agent, as discussed in more detail below. It will be recognized by those skilled in the art, that no physical transformation of the enzyme may actually occur when the enzyme is converted from the first state into the second state, or vice versa. That is, when the enzyme is converted from the first state into the second state, the chemical composition of the enzyme typically does not change. Without wishing to be bound by any particular scientific theory, it is believed that upon conversion of the enzyme from the second state into the first state, a conformational change occurs such that the enzyme adopts the correct active site geometry which can bind to, and digest, nucleic acid.

As used here “activator” refers to any species which upon binding to the enzyme converts the enzyme into the first state or to active enzyme. Preferably, the activator is a small molecule (e.g. <5 kDa), a metal ion or a protein. The activator typically binds to the enzyme reversibly, such that removal of the activator induces the enzyme to convert back to its second state, which has minimal activity. Depending on the nature and properties of the enzyme, one skilled in the art given the benefit of this disclosure will be able to select suitable activators. Enzymes that can be selectively activated by cations in solution are known. Most DNA metabolizing enzymes or nucleases require magnesium for activity. However, some nucleases are activated by calcium ions rather than magnesium ions. One such example is micrococcal nuclease from Staphylococcus aureus which has a molecular mass of about 16.8 kDa. Micrococcal nuclease is a well-known enzyme that catalyzes cleavage of both DNA and RNA to yield 3′-nucleotides and oligonucleotides. See Alexander, M., Heppel, L. A. and Hurwitz, J., J. Biol. Chem. 236, 3014 (1961), the entire disclosure of which is incorporated herein by reference for all purposes. However, selectively activatable nucleases and accordingly, selectively inactivatable nucleases have not been used previously to remove contaminating nucleic acids in reagent solutions. Such nucleases provide for a rapid method of removing contaminating DNA from reagents without requirement of further purification techniques which can be time consuming and expensive.

As used here “inactivating agent” refers to any species which can convert the enzyme into the second state or any species which can force the enzyme to maintain the second state, i.e. the selectively inactivatable enzyme. Preferably, inactivating agents bind to and remove the activators such that the enzyme is forced to adopt the second state. In other embodiments, the inactivating agents themselves bind to the enzyme and force the enzyme into the second state. Depending on the nature and properties of the enzyme, one skilled in the art given the benefit of this disclosure will be able to select suitable inactivating agents.

As used here “incubation period” refers to the amount of time where the enzyme is allowed to digest contaminating nucleic acid. Preferably, incubation period refers to the amount of time where the enzyme is in its first state having maximal enzymatic activity. The incubation period typically will vary with the kinetic properties of the enzyme. Depending on the nature and properties of the enzyme, one skilled in the art given the benefit of this disclosure will be able to select suitable incubation periods.

As used here, “selectively activatable” or “selectively activated” refers to the condition where the enzyme is functional to degrade, to digest, or otherwise render inert, one or more nucleic acids or nucleic acid fragments.

As used here, “selectively inactivatable” or “selectively inactivated” refers to the condition where the enzyme is not operative, e.g. not functional. That is “selectively inactivated” refers to the condition where the enzyme has substantially no catalytic activity, e.g. less than 1% of contaminating nucleic acid is digested when the enzyme is rendered selectively inactivatable.

As used here “render inert” refers to the altering or modifying of nucleic acids(s) such that the nucleic acid(s) cannot interfere with any subsequent chemical and/or biological reactions. The nucleic acid(s) can be rendered inert by chemical modification of the nucleic acid, e.g. by removal of one or more functional groups such that the nucleic acid is unable to react with a DNA template or polymerase, for example. Depending on the nature of the enzyme selected, the mechanism by which the nucleic acid is rendered inert can vary. For example, in embodiments where the enzyme comprises micrococcal nuclease from Staphylococcus aureus, the nucleic acid is rendered inert by leaving free nucleic acids or nucleic acid fragments having a 3′-phosphate group and a 5-hydroxyl group. Such nucleic acids are referred to in some instances below as “inert free nucleic acids.”

In accordance with certain preferred embodiments, a method of rendering nucleic acid inert, whether the nucleic acid is contaminating nucleic acid or added nucleic acid, is disclosed. An enzyme is added to a solution, the enzyme having maximal activity in the presence of bound activator and minimal activity in the absence of bound activator and with the enzyme capable of rendering nucleic acid inert in the presence of bound activator and incapable of rendering nucleic acid inert in the absence of bound activator. The enzyme having bound activator is incubated in a manner to allow the enzyme to render the nucleic acid inert. The bound activator is removed to inhibit the enzyme from further acting on nucleic acid. Preferably, the enzyme having bound activator acts to render the nucleic acid inert by degrading, e.g. digesting, the nucleic acid to yield phosphate moieties at the 3′ ends and hydroxyl moieties at the 5′ ends of nucleotides or nucleotide fragments. However, other methods of rendering the nucleic acids chemically inert are possible. For example, one or more groups of the nitrogenous bases could be modified such that little or no hydrogen bonding can occur, thus preventing hybridization of the degraded nucleic acid or nucleic acid fragments to a DNA template, for example. In certain preferred embodiments, the enzyme for digesting nucleic acids preferably is a micrococcal nuclease, such as the micrococcal nuclease from Staphylococcus aureus. In embodiments where the enzyme is micrococcal nuclease, preferably the activator is calcium ions and the calcium ions can be removed by any compound or method which provides for the dissociation of the calcium ions from the enzyme. Preferably, the calcium ions dissociate from the enzyme and is sequestered such that it cannot bind to the enzyme again. Suitable agents for sequestering calcium ions, i.e. suitable sequestering agents, include but are not limited to EDTA, EGTA, BAPTA and the like. Preferably, the agent which sequesters calcium ions binds much tighter to calcium ions than the enzyme does. Therefore, addition of the sequestering agent can remove the calcium ions from the enzyme, can keep the calcium ions from rebinding to the enzyme, and thus can inhibit the activity of the enzyme, i.e. can prevent the enzyme from further digesting nucleic acid. It will be within the ability of those skilled in the art given the benefit of this disclosure to select suitable concentrations of micrococcal nuclease, calcium ions and sequestering agents in accordance with the methods disclosed here.

In accordance with certain preferred embodiments, after digestion with the enzyme, the extent of digestion can be tested using gel electrophoresis, mass spectroscopy, or other suitable techniques. If the contaminating nucleic acid has not been sufficiently degraded such that it will not interfere with subsequent analyses and methods, additional activator can be added to allow the enzyme to bind to the additional activator and digest remaining contaminating nucleic acid. Preferably, an effective amount of additional activator is added such that substantially all enzyme present has bound activator. After incubation for a suitable period to allow for digestion of remaining contaminating nucleic acid, the additional bound activator can be removed, for example, by adding additional sequestering agent. The additional sequestering agent will remove the bound activator and thus inhibit the enzyme from further acting on nucleic acid. One skilled in the art given the benefit of this disclosure will be able to add effective amounts of additional activator to allow for removal of additional contaminating nucleic acids.

In accordance with certain preferred embodiments, once substantially all the contaminating nucleic acid has been rendered inert, a DNA template, a DNA polymerase, primers and optionally free nucleotides if not already present can be added to the solution to amplify the DNA template. No separation of the enzyme or degraded contaminating nucleic acids from the solution is necessary since the enzyme in inactive and the degraded contaminating nucleic acids have been rendered inert and cannot interfere with the DNA synthesis reaction. Preferably the DNA template is either a single-stranded DNA template or a double-stranded DNA template. Preferably the DNA polymerase is any polymerase which is operative at high temperatures, such as those commonly used in polymerase chain reaction, or is any DNA polymerase which is commonly used to perform isothermal DNA amplification methods. The primers typically depend on the sequence of the DNA template to be amplified, and one skilled in the art given the benefit of this disclosure will be able to design and select suitable primers depending on the sequence and nature of the DNA template to be amplified. Preferably, the free nucleotides include but are not limited to dATP, dGTP, dCTP and dTTP. Other components, such as suitable buffers, salts, and the like may also be added to allow the DNA amplification to occur efficiently. The amplification of the DNA template may be performed using thermal cycling methods known to those skilled in the art or can be performed using isothermal DNA amplification methods known to those skilled in the art. In embodiments where thermal cycling is used, the heating step typically used to denature a double-stranded DNA template can also denature the enzyme used to remove contaminating nucleic acid, thus rendering the enzyme inactive. Therefore, as a precautionary measure one or more cycles of heating and cooling can be performed prior to the addition of DNA template to be amplified and/or free nucleotides such that the enzyme will be rendered inactive prior to amplifying the DNA. In certain embodiments, the sequestering agent is omitted and the heating and cooling steps are used to inactivate the enzyme. That is, in certain embodiments, it is not necessary to remove the activator because the denatured enzyme, even with bound activator, cannot degrade nucleic acids. One skilled in the art given the benefit of this disclosure will be able to select suitable conditions for thermally cycling the solution to provide for amplification of a DNA template and/or to render the enzyme inactive.

In accordance with certain preferred embodiments, a method of digesting contaminating nucleic acid is disclosed that includes adding an enzyme to a solution for performing a DNA synthesis reaction. The enzyme is capable of digesting the contaminating nucleic acid in a first state and incapable of digesting the contaminating nucleic acid in a second state. The enzyme is converted into the first state by adding an activator. The enzyme in the first state is then incubated in a manner to allow for digestion of the contaminating nucleic acid by the enzyme in the first state. The enzyme is then converted to the second state by adding an inactivating agent. The enzyme may be added to the solution using standards methods, e.g. micropipettes. In embodiments, where the DNA synthesis reaction is automated, the enzyme can be automatically added to the reaction by the instrument. It will be recognized by those skilled in the art given the benefit of this disclosure that the enzyme and activator can be added simultaneously to the solution. That is, two separate steps are not necessary to convert the enzyme to the first state. The enzyme typically can be converted to the first state prior to adding enzyme to the solution by adding activator directly to enzyme and then adding the combination to the solution for performing a DNA synthesis reaction. The inactivating agent may be added using similar methods used to add the activator. Preferably, the inactivating agent is added after a suitable incubation period such that the enzyme can digest substantially all contaminating nucleic acid, e.g. >99.9% of the contaminating nucleic acid. Without wishing to be bound by any particular scientific theory, in certain embodiments the inactivating agent can bind to the activator much tighter than the activator can bind to the enzyme. The result is that the activator is stripped from the enzyme and binds to the inactivating agent. Preferably the dissociation constant for inactivating agent for the activator is less than about 10⁻⁹ M⁻¹, for example. However, as long as the dissociation constant for the inactivating agent is lower than, e.g. 10× lower than, the dissociation constant of the activator the inactivating agent can strip the activator from the enzyme. Thus, depending on the nature of the enzyme, activator and inactivating agent, the dissociation constants may vary. When the activator is removed from the enzyme, the enzyme is converted into the second state such that substantially no further digestion of nucleic acid occurs. Because, the enzyme has been rendered inactive by removal of the activator, it is unnecessary to remove the enzyme from the solution for performing a DNA synthesis reaction. That is, no purification steps are necessary prior to using the solution for performing the DNA synthesis reaction. It is a significant advantage that embodiments of the present invention provide for removal of contaminating nucleic acids without the need to separate the digestion products prior to performing a DNA synthesis reaction.

In accordance with certain preferred embodiments, the enzyme for digesting contaminating nucleic acids preferably is a micrococcal nuclease, such as the micrococcal nuclease from Staphylococcus aureus. In embodiments, where the enzyme is micrococcal nuclease, preferably the activator is calcium ions and the inactivating agent is EDTA, EGTA, or other reagent which binds tightly to calcium ions, i.e. which binds tighter to calcium ions than the micrococcal nuclease. Without wishing to be bound by any particular scientific theory, it is believed that binding of calcium ions by the micrococcal nuclease converts the micrococcal nuclease into an active form, e.g. an enzymatically active form. Removal of the bound calcium ions typically also removes the enzymatic activity. That is, when the bound calcium ions are removed from the micrococcal nuclease, the micrococcal nuclease has substantially no enzymatic activity. Thus, addition of calcium ions to the enzyme converts the enzyme into the first state, and subsequent removal of the bound calcium ions from the enzyme, by an inactivating agent such as EDTA, converts the enzyme into the second state. It will be within the ability of those skilled in the art given the benefit of this disclosure to select suitable concentrations of micrococcal nuclease, calcium ions and inactivating agent in accordance with the method disclosed here.

In accordance with certain preferred embodiments, the enzyme may also be an enzyme which is normally active, e.g. natively active in the first state. Thus, no activator is necessary to convert the enzyme into the first state. However, binding of an inactivating agent to the enzyme directly, e.g. binding of a protein to the enzyme, can convert the enzyme into the second state. For example, certain proteins can be constructed, using the methods described below, for example, which bind irreversibly to the active site of the enzyme, thus rendering the enzyme unable to digest any nucleic acids. In other embodiments, irreversible inhibitors are added to the enzyme such that the enzymatic activity is reduced to minimal levels or substantially no activity. Depending on the nature and properties of the enzyme, one skilled in the art given the benefit of this disclosure will be able to select and to design suitable inactivating agents for converting the enzyme from a first state into a second state.

In accordance with certain preferred embodiments, a DNA template, primers, and optionally free nucleotides if not already present can be added to the solution after the enzyme is converted to the second state. That is, once all contaminating nucleic acid is digested and the enzymatic activity has been reduced to minimal levels, i.e. by converting the enzyme into the second state, reagents which are necessary to amplify a DNA template, e.g. a single or double-stranded DNA template, can be added to the solution for performing a DNA synthesis reaction, such as the polymerase chain reaction or isothermal amplification methods. No purification steps are required prior to addition of the reagents for performing the DNA synthesis reaction. Suitable reagents and concentrations for performing the DNA synthesis reactions will be readily apparent to those skilled in the art given the benefit of this disclosure.

In accordance with certain preferred embodiments, a method is provided in which nucleic acids are altered by digestion in a manner to prevent their further reactivity in nucleic acid amplification methods. That is, nucleic acids which are present are modified, e.g. chemically modified, or altered such that the products of the nucleic acids (referred to in some instances below as altered nucleic acid products) do not interfere with any subsequent chemical or biological reactions taking place. Because the nucleic acids do not interfere with subsequent chemical reactions, e.g. such as those performed during PCR and/or isothermal DNA amplification methods, it is not necessary to remove the altered nucleic acid products from the reagents or solutions prior to performing subsequent assays. That is, no separation steps are required prior to performing chemical or biological assays, e.g. PCR, or prior to performing analytical techniques, e.g. mass spectroscopy such as ESI-MS, MALDI-TOF-MS, FAB-MS and the like. Accordingly, the total time required to perform analyses such as PCR or isothermal DNA amplification methods is reduced, because the time required to remove any contaminating nucleic acids from solutions and/or reagents is avoided. One skilled in the art, given the benefit of this disclosure, will be able to use the methods disclosed here for removal of unwanted nucleic acids which may be present in solutions and/or reagents.

In accordance with certain preferred embodiments, at least one enzyme is provided which under a first condition is operative to render contaminating nucleic acids unable to interfere with subsequent assays involving nucleic acids and under a second condition is incapable of altering any nucleic acids which are present. For example, enzymes useful in the practice of the present invention include those that can be selectively activated to alter or to digest nucleic acids and/or selectively inactivated to prohibit or to cease nucleic acid digestion or alteration. In preferred embodiments, the enzyme chemically modifies the nucleic acid in a manner to prevent the nucleic acid from reacting with other species which may be subsequently added to the solution or mixture, e.g. dNTPs. Preferably, the enzyme modifies the nucleic acids such that the nucleic acids or nucleic acid fragments have a 3′-phosphate group and a 5′-hydroxyl group. One particular enzyme, which is capable of modifying nucleic acids to have a 3′-phosphate group and a 5′-hydroxyl group is micrococcal nuclease from Staphylococcus aureus. The micrococcal nuclease is activated to digest nucleic acids by the addition of one or more activators, such as calcium ions, for example. The activated micrococcal nuclease can digest any DNA, or RNA, that is present, leaving free nucleic acids or nucleic acid fragments having a 3′-phosphate group and a 5′-hydroxyl group. Since DNA or RNA containing a 3′-phosphate group is unable to function as a primer to initiate DNA or RNA synthesis, the products formed by digestion with micrococcal nuclease are generally inert, i.e. rendered inert. That is, the products formed are not reactive with any DNA templates that may be added to the solution, and the products also cannot function as primers for amplification. This result is a significant advantage over nucleases that leave products with 3′ hydroxyl groups, e.g. DNase I, that produce partial products that will efficiently prime nucleic acid synthesis and thus can produce a potent source of contaminating amplification. Therefore, increased control, purity, and efficiency are gained by using the methods described here. One skilled in the art given the benefit of this disclosure will be able to select suitable concentrations and conditions for exposing any nucleic acids to suitable enzymes, e.g. micrococcal nuclease, for rendering the nucleic acids inert.

In accordance with certain preferred embodiments, the enzyme is added in an effective amount. The effective amount typically depends on the catalytic and/or kinetic properties of the enzyme, e.g. k_{cat}, K_{M}, K_{D} and the like. Preferably enough enzyme is added such that the enzyme is not saturated with nucleic acid. Alternatively, enough enzyme can be added such that greater than about 90% of the nucleic acid is rendered inoperative during the incubation period. Preferably greater than 95%, e.g. greater than 99%, of the nucleic acid is rendered inoperative during the incubation period. One skilled in the art given the benefit of this disclosure will be able to select suitable amounts and/or concentrations of enzyme for use in the methods disclosed here.

In accordance with certain preferred embodiments, the at least one enzyme can be activated by addition of one or more activators. One skilled in the art given the benefit of this disclosure, will recognize that the choice of activator depends on the enzyme selected for use in rendering the nucleic acids inert. That is, the activator is typically selected based on the properties and characteristics of the enzyme that is chosen. In embodiments where micrococcal nuclease is selected, calcium ions are typically added to activate the micrococcal nuclease. It will be recognized by those skilled in the art that divalent metal species which can functionally substitute for calcium ions could also be used. The activator can be added to the enzyme to activate the enzyme prior to adding the enzyme to the solution to remove any contaminating nucleic acids, or the activator and the enzyme can be added simultaneously to the solution to remove any contaminating nucleic acids. One skilled in the art given the benefit of this disclosure will be able to select suitable activators depending on the enzyme which is selected.

In accordance with certain preferred embodiments, to prevent the active enzyme from digesting any desired DNA products, e.g. products from PCR or isothermal methods, one or more enzyme inactivating agents can be added to quench the activity of the enzyme to digest nucleic acids prior to addition of the nucleic acids necessary for amplification of a DNA template. According to one embodiment, micrococcal nuclease inactivating agents are compounds capable of removing calcium ions, which is required for enzymatic activity. Suitable inactivating agents include ethylenediaminetetraacetic acid (EDTA) and ethylene glycol-bis(2-aminoethyl ether)-N,N,N′,N′-tetraacetic acid (EGTA). In alternative embodiments, the bound calcium ions may be displaced by addition of a species having a higher binding affinity to the calcium ion sites of the micrococcal nuclease. Suitable species known to displace calcium ions and typically inactivate enzymes include divalent and trivalent transition metal species, including but not limited to Mn²⁺, Zn²⁺, Ni²⁺, Cr³⁺, Sr²⁺, and trivalent lanthanide species, including but not limited to La³⁺ and Gd³⁺. Preferably, the divalent and trivalent metal species can bind to the calcium ion sites but cannot functionally replace the calcium ions. That is, the divalent and trivalent species can bind to the calcium ion sites but the enzyme is not active when the divalent and trivalent species are bound to the enzyme. Without wishing to be bound by any scientific theory, it is believed that these species bind with higher affinity than calcium ions because of increased charge:size density. Because many of these divalent and trivalent species have empty or partially filled orbitals, e.g. empty or partially filled d- and f-orbitals, the three-dimensional active site geometry of the enzyme, found in the presence of bound calcium ions, is disrupted by interactions between the protein and the empty orbitals of the metal ion replacing the bound calcium ions. Therefore, it is believed that these species displace bound calcium ions and distort the active site geometry to such a degree that enzymatic activity is reduced if not inhibited. These divalent metal species may be used in combination with the EGTA or EDTA treatment to provide increased certainty that the enzymatic activity has been removed. That is, the divalent and trivalent metal species may be added prior to, together with, or subsequent to addition of EGTA and EDTA. In certain preferred embodiments, the concentrations of EDTA and EGTA are approximately at least one-half the concentration of calcium ions used to activate the micrococcal nuclease, since it is well known that one EDTA molecule can bind two calcium ions molecules. In preferred embodiments, the concentrations of EDTA and EGTA are equal to or greater than the calcium ion concentrations used to activate the enzyme, e.g. the concentrations of EGTA and EDTA are 2×, 5× or 10× greater than the concentration of calcium ions used. In embodiments, where divalent and trivalent metal species are used to inactivate the enzyme, the concentration of EDTA or EGTA is adjusted to account for binding of the divalent and trivalent metal species to the EDTA or EGTA species. One skilled in the art, given the benefit of this disclosure will be able to select concentrations of calcium ions and micrococcal nuclease inactivating agents suitable for an intended use.

In accordance with certain preferred embodiments, inhibitors which bind to the enzyme in a manner to inhibit its ability to digest nucleic acids or reagents which otherwise degrade the enzyme to prevent further nucleic acid digesting activity of the enzyme can also be used. Methods for the selection of DNA aptamers that bind to and inhibit Taq DNA polymerase include Lin, Y., Jayasena S. D., J. Mol. Biol. Aug. 8, 1997:271(1): 100-111, the entire disclosure of which is incorporated herein by reference in its entirety for all purposes, the teachings of which could be used to select for DNA or RNA aptamers that bind to and inhibit micrococcal nuclease or other enzymes within the scope of the present invention. Similarly, methods for the use of an antibody to bind to and inhibit Taq DNA polymerase include Kellog, D. E., Rybalkin, I., Chen, S., Mukhamedova, N., Vlasik, T., Siebert, P. D., and Chenchik, A., Biotechniques June 1994; 16(6): 1134-7, the entire disclosure of each of which is incorporated herein by reference for all purposes, the teachings of which could be used to select for DNA or RNA antibodies that bind to and inhibit micrococcal nuclease or other enzymes useful in the methods of the present invention. Preferably, the use of certain aptamers and antibodies as inhibitors are applicable in isothermal amplification techniques to minimize any dissociation which may result from high temperatures. One skilled in the art given the benefit of this disclosure will be able to select and/or to design suitable inhibitors for binding to the selectively activatable enzymes used in the methods disclosed here.

In accordance with certain preferred embodiments, a selectively activatable enzyme is used to digest contaminating nucleic acids after which the solution containing the enzyme can be heated to denature the enzyme rendering it inactive. Suitable temperatures for denaturing the enzyme will be apparent to those skilled in the art given the benefit of this disclosure. Denaturation of the enzyme typically removes an enzymatic activity. In certain embodiments where micrococcal nuclease is used as the enzyme, the heating procedure is used in combination with a chelating agent, such as EDTA or EGTA. This combination provides for denaturation as well as removal of any calcium ions which may be present in solution or bound to the enzyme. The combination of the two procedures can ensure that the micrococcal nuclease was completely inactivated.

In accordance with certain preferred embodiments, the methods disclosed here are suitable for use in DNA synthesis reactions. For example, a selectively activatable enzyme can be added to a solution comprising contaminating nucleic acid, an activator can be added to activate the enzyme, the solution can be incubated for a suitable time to allow digestion of the contaminating nucleic acids by the enzyme, the enzyme can be deactivated using a suitable inactivating agent, and a DNA template and any necessary primers, dNTPs, etc. can be added to amplify the DNA template. The apparatus, e.g. micropipette tips and the like, used to add the DNA template and dNTPs to the solution can also be treated with the enzyme to remove any contaminating nucleic acids. In certain embodiments, standard DNA amplification and synthesis techniques may be performed, such as the DNA amplification techniques described in U.S. Pat. Nos. 4,683,195, 4,683,202, 6,143,531, and 4,994,370, the entire disclosure of each of which is incorporated herein by reference for all purposes. These DNA amplification and synthesis methods include but are not limited to thermally cycled methods, such as PCR, and isothermal methods, such as those based on T7 DNA replication systems. Preferably, all buffer solutions and reagents are treated with the activatable enzyme such that no contaminating nucleic acids are present. This process allows amplification of minute quantities of DNA without contamination by unwanted nucleic acids. In certain preferred embodiments, amplification systems in general can be divided into specific and nonspecific systems. Specific amplification systems are primer-based. They either involve temperature cycling (PCR) or are isothermal. Examples of specific, isothermal amplification systems include self-sustained sequence replication systems (Gebinoga M, Oehlenschlager F, Eur J Biochem Jan. 15, 1996;235(1-2):256-61) and strand displacement amplification (Nadeau J G, Pitner J B, Linn C P, Schram J L, Dean C H, Nycz C M, Anal Biochem. Dec. 15, 1999;276(2):177-87, and rolling circle amplification (Lizardi P M, Huang X, Zhu Z, Bray-Ward P, Thomas D C, Ward D C. Nat Genet. July 1998;19(3):225-32), the entire disclosure of each of which is incorporated herein by reference for all purposes. Additional isothermal amplification systems especially useful in the practice of the present invention are described in WO 00/41524, the entire disclosure of which is incorporated herein by reference for all purposes. The self-sustained sequence replication system is based on the production of RNA while the strand displacement amplification and rolling circle amplification methods produce DNA. The methods disclosed here are suitable for use in any of these methods to reduce the amount of contaminating nucleic acid which is present. One skilled in the art given the benefit of this disclosure will be able to use the methods disclosed here for reducing contaminating amounts of nucleic acids in any assay or reaction involving nucleic acids.

Recently, a whole genome isothermal amplification procedure has been developed using bacteriophage Ø29 DNA polymerase and random hexamers. See Dean, F. B., Hosono, S., Fang, L., Wu, X., Faruqi, A. F., Bray-Ward, P., Sun, Z., Zong, Q., Du, Y., Du, J., Driscoll, M., Song, W., Kingsmore, S. F., Egholm, M., and Lasken, R. S. (2002) Proc. Natl. Acad. Sci. USA 99, 5261-5266 (hereby incorporated by reference in its entirety) which describes the use of Ø29 DNA polymerase and random hexamers in a whole genome amplification procedure, and Dean, F. B., Nelson, J. R., Giesler, T. L., and Lasken, R. S. (2001) Genome Res. 11, 10951099 (hereby incorporated by reference in its entirety), which describes the use of Ø29 DNA polymerase and random hexamers to amplify small amounts of plasmid DNA. In a preferred embodiment, the methods described in this application would be used with these amplification procedures to remove contaminating DNA from the reaction mixture before adding the DNA to be amplified.

In accordance with certain preferred embodiments, a method for amplifying DNA is disclosed. The method comprises treating one or more reagent solutions with an effective amount of a selectively activatable and/or inactivatable enzyme that is capable of digesting any contaminating nucleic acids, e.g. digesting any contaminating DNA and/or RNA. Preferably the reagent solutions are those commonly used to perform PCR including solutions comprising a DNA polymerase, one or more buffer solutions, one or more salt solutions, free nucleotides (dNTPs), primers, etc. To activate the enzyme, an activator, such as calcium ion, is added to the solution. However, the activator can be added at the same time as the enzyme and the other ingredients in solution or it can be added to the enzyme alone to produce an activated enzyme which is then added to the ingredients. That is, the enzyme need not be activated by a separate step of adding an activator. The active enzyme digests any contaminating nucleic acids which may be introduced into solution by any of the individual ingredients, such as the dNTPs. However, enzymes such as micrococcal nuclease typically do not alter the dNTPs. The resulting products of digestion of contaminating nucleic acids are incapable of being used as primers for DNA synthesis because they do not contain 3′ hydroxyl groups. An inactivating agent, such as EGTA, is added to the solution to inactivate the enzyme. Without wishing to be bound by any scientific theory, it is believed that the EGTA binds to the calcium ions with a higher binding affinity than the affinity at which the enzyme binds calcium ions. Such binding affinities, or dissociation constants as the case may be, of EGTA for Ca²⁺ are well known to those skilled in the art or can be determined using binding analyses, e.g. Scatchard analysis. After removal of any calcium ions that is bound to the enzyme by addition of EGTA, or other suitable chelating agent, a single-stranded or double-stranded DNA template, nucleotide primers (if appropriate for the particular amplification method chosen), and optionally free nucleotides are added to the solution without the need to separate the inactivated enzyme from the solution. The DNA template may comprise any DNA template desired for amplification purposes. Design of nucleotide primers are well known to those skilled in the art and suitable methods for designing nucleotide primers may be found in U.S. Pat. Nos. 4,683,195, 4,683,202, 4,942,130, 4,946,786, 5,639,608, 6,143,531, and 4,994,370, the entire disclosures of each of which are incorporated herein by reference for all purposes. The free nucleotides preferably comprise dGTP, dATP, dTTP, and dCTP. For amplification systems based on RNA synthesis, rNTPs would be added. For T7-based isothermal amplification systems, both 4 dNTPs and 4 rNTPs are added. Suitable primer concentrations can be selected by those skilled in the art given the benefit of this disclosure. After addition of the DNA template, and the nucleotide primers if appropriate, the solution can be thermally cycled using heating and cooling steps. The solution can then heated to a sufficient temperature to allow for denaturation of the double stranded DNA template. The temperature is then cooled to allow for annealing of the primers and subsequent binding of the DNA polymerase. The primers annealed to each of the single strands of the DNA are elongated, using the single strands as templates, to yield two identical strands of double-stranded DNA. These steps of denaturing the DNA, annealing the primers, and strand elongation is repeated for a sufficient number of cycles to provide a suitable quantity of DNA. One skilled in the art given the benefit of this disclosure will be able to select suitable temperatures, conditions, and number of cycles to produce a suitable quantity of DNA.

In accordance with certain preferred embodiments, a kit is provided comprising a selectively activated enzyme, an enzyme activator, and a chelating agent or a metal species capable of displacing the activator, or both. In preferred embodiments, the selectively activated enzyme comprises micrococcal nuclease, the enzyme activator comprises calcium ions, and the chelating agent comprises EDTA or EGTA. The kit may be used for removal of contaminating nucleic acids in test tubes, pipette tips, Eppendorf® tubes, and other vessels and apparatus used in nucleic acid research, nucleic acid amplification procedures, nucleic acid sequencing procedures, forensic applications, and the like. Such treatment may be used in combination with currently used sterilization techniques such as autoclaving, etc. Typically the enzyme and the activator are mixed together to activate the enzyme. Preferably an excess of activator is added, e.g. 10×, 20× or 100× greater than the K_{D} of the activator, is added to ensure that the maximum amount of enzyme is active. The solution of activated enzyme is typically then added to the apparatus or other solutions and allowed to incubate for a suitable time to digest, or render inoperative, any contaminating nucleic acids. Suitable incubation temperatures will be readily selected by those skilled in the art given the benefit of this disclosure and typically the incubation temperatures depend on the activity profile (with respect to temperature and substrate concentration) of the enzyme. In embodiments where micrococcal nuclease is used, preferably the incubation temperature is about 37° C. The incubation period typically is any time which provides for suitable alteration of at least about 90% of the contaminating nucleic acids. More preferably, the incubation period is long enough to allow at least about 95%, and more preferably at least about 99%, of the contaminating nucleic acid to be digested or rendered inert. One skilled in the art given the benefit of this disclosure will be able to select suitable incubation periods for use in the methods disclosed here.

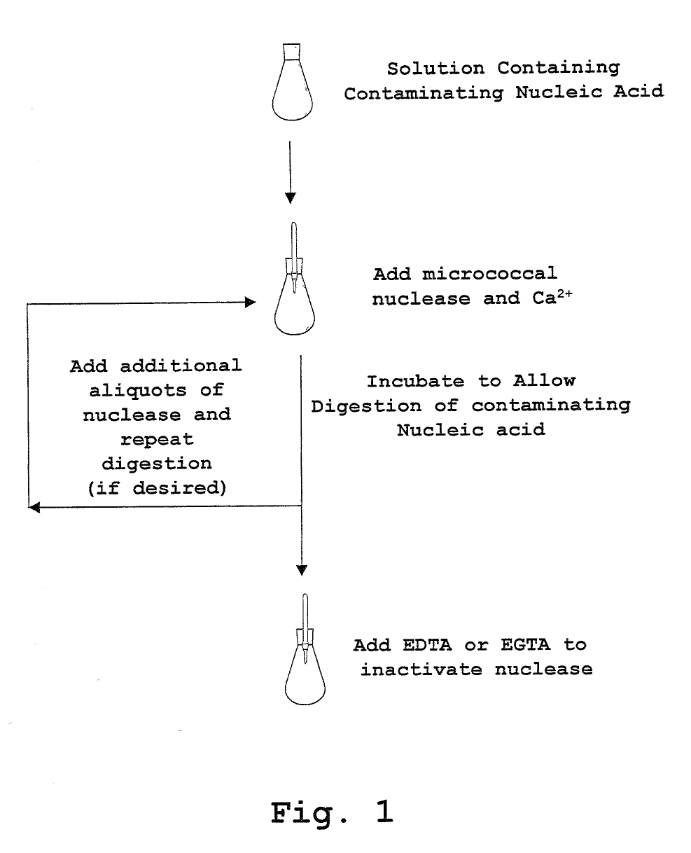

In accordance with certain preferred embodiments, and referring to FIG. 1, a method of removing contaminating nucleic acid comprises adding a selectively activatable and/or inactivatable enzyme, such as a micrococcal nuclease, to a solution comprising contaminating nucleic acid. Such solutions may include PCR reagents, buffers, salt solutions, free nucleotides (whether dNTPs or rNTPs), polymerases and other enzymes such as binding proteins and helicases for example to be used with T7-based isothermal amplification systems. In embodiments comprising a micrococcal nuclease, the nuclease is added to the solution and typically is not active in the absence of an activator. To activate the micrococcal nuclease, an activator, such as calcium ions, is added to the micrococcal nuclease. According to an alternative embodiment, the activator and the enzyme are combined to activate the enzyme prior to being added to the solution or the activator and the enzyme can be added to the solution at the same time. While not wishing to be bound by any particular scientific theory, it is believed that upon binding to calcium ions, the micrococcal nuclease is activated and can digest nucleic acids, for example, contaminating nucleic acids, present in the solution. It is a significant advantage that the digestion products have a 3′-phosphate group and a 5′-hydroxyl group. These digestion products are incapable of interfering with subsequent amplification of DNA, and, thus, there is no need to separate the digestion products from the reagents prior to addition of any DNA templates and primers, where appropriate, to the solutions. After incubation for a suitable time, e.g. 60 minutes at 37° C., to allow digestion of any contaminating nucleic acid, the enzyme is inactivated by addition of one or more inactivating agents. In preferred embodiments, the inactivation occurs by addition of a chelating agent such as EDTA or EGTA, or both, to remove any bound Ca²⁺ ions from the micrococcal nuclease. Therefore, because the micrococcal nuclease is rendered inactive, there typically is no need to separate the micrococcal nuclease from the other species in the reagent solutions prior to addition of DNA templates and primers where appropriate, to the solutions for amplification of DNA. Because the activated micrococcal nuclease digests any contaminating DNA or RNA, very minute amounts of DNA can be amplified with great accuracy using the methods described here and the standard PCR methods and isothermal DNA amplification methods known to those skilled in the art.

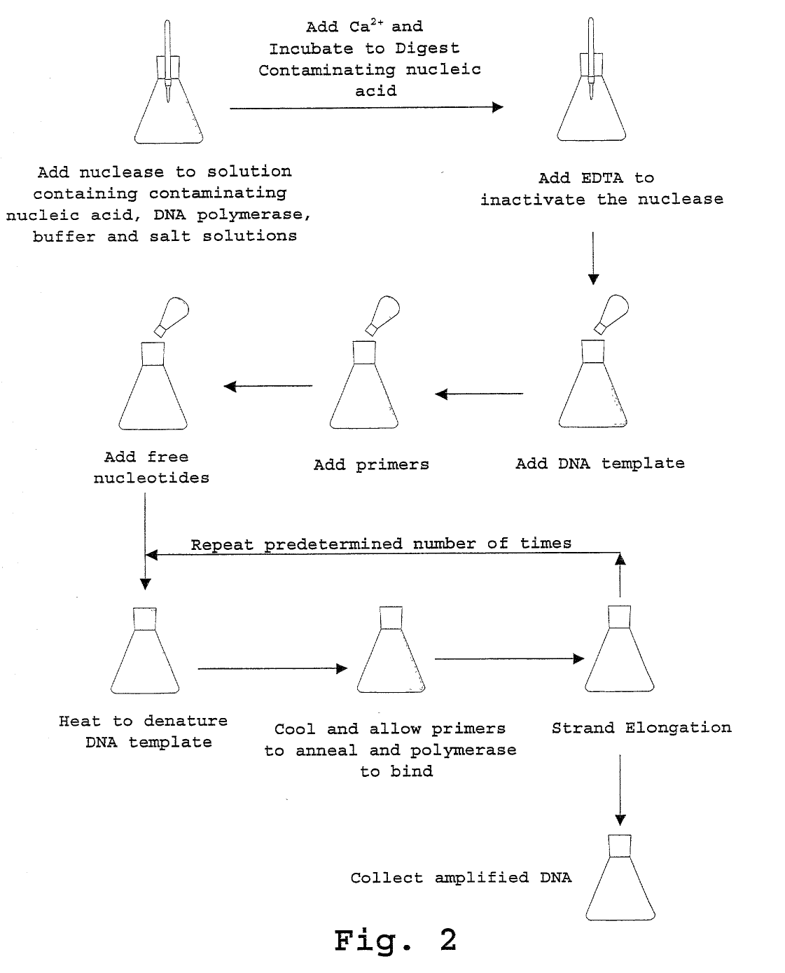

In accordance with certain preferred embodiments, and referring to FIG. 2, after digestion of any contaminating nucleic acids, the solutions may then be used to perform any of the DNA amplification or synthesis techniques well known to those skilled in the art. In certain embodiments, PCR is used to amplify the DNA. Suitable methods for performing PCR are well-known to those skilled in the art, such as the methods disclosed in Molecular Biology Techniques Manual, 3^{rd} Ed., edited by Vernon E. Coyne, M. Diane James, Sharon J. Reid and Edward P. Rybicki.

In certain preferred embodiments, and referring to FIG. 2, the method disclosed here is used in PCR by treatment of Taq polymerase (or Pfu polymerase or other DNA polymerase) in a suitable buffer, typically comprising at least Tris-HCl, K⁺ and Mg²⁺, with the micrococcal nuclease and calcium ions and free nucleotides e.g. all four dNTPs (dGTP, dATP, dTTP, and dCTP). After incubation for a suitable time, preferably at least 30 minutes at 37° C., EDTA or EGTA is added to remove any bound calcium ions and thus deactivate the micrococcal nuclease to prevent further nucleic acid digestion. The amount of Mg²⁺ in the buffer solution is typically adjusted to account for the addition of the EGTA and EDTA, thus ensuring that the polymerase remains active. As FIG. 2 indicates, nucleotide primers, preferably at a total concentration of about 0.2-1.0 μM (preferably at a concentration of about 50-200 μM each) and the target DNA template (preferably about 1 ng to 1 μg) is added to the reagent solution that has been treated with the micrococcal nuclease. Free nucleotides can also be added after inactivation of the nuclease. Other reagents, such as DMSO, glycerol, betaine, DNA binding proteins and other additives used by those skilled in the art for performing PCR, may optionally be added to the solution. The thermal cycling may be performed using any standard thermal cycler apparatus, such as those available from MJ Research, Inc, Waltham, Mass., e.g. the PTC-100 thermal cycler. It will be within the ability of those skilled in the art to adjust the thermal cycling parameters, e.g. the denaturation, annealing, and cooling times and temperatures, for an intended use and for an intended amount of DNA product. For example, in certain embodiments, the initial denaturation step occurs by heating the entire solution to about 95° C. for about 5 minutes. Annealing of the primers and subsequent binding of the polymerase to the DNA/primer complex occurs at about 50-70° C. for about 1 minute. Elongation of each DNA strand occurs at about 72° C. for about 1 minute. This process is typically repeated for 25-40 cycles to amplify the DNA template. After the last cycle, incubation for another 10 minutes at 72° C. is performed to complete both strands. Because any contaminating DNA has been removed with the micrococcal nuclease treatment, the end product of the amplification process is multiple copies of the added DNA template without any substantial amplification of contaminating nucleic acids. Other suitable PCR protocols and PCR reagents known to those skilled in the art, such as those available from Abgene, Inc., Surrey, UK, and those described in U.S. Pat. Nos. 4,683,202, 4,683,195, 4,965,188, 5,075,216, and 5,616,3011, the entire disclosure of each of which is incorporated herein by reference for all purposes, will be readily selected by those skilled in the art given the benefit of this disclosure.

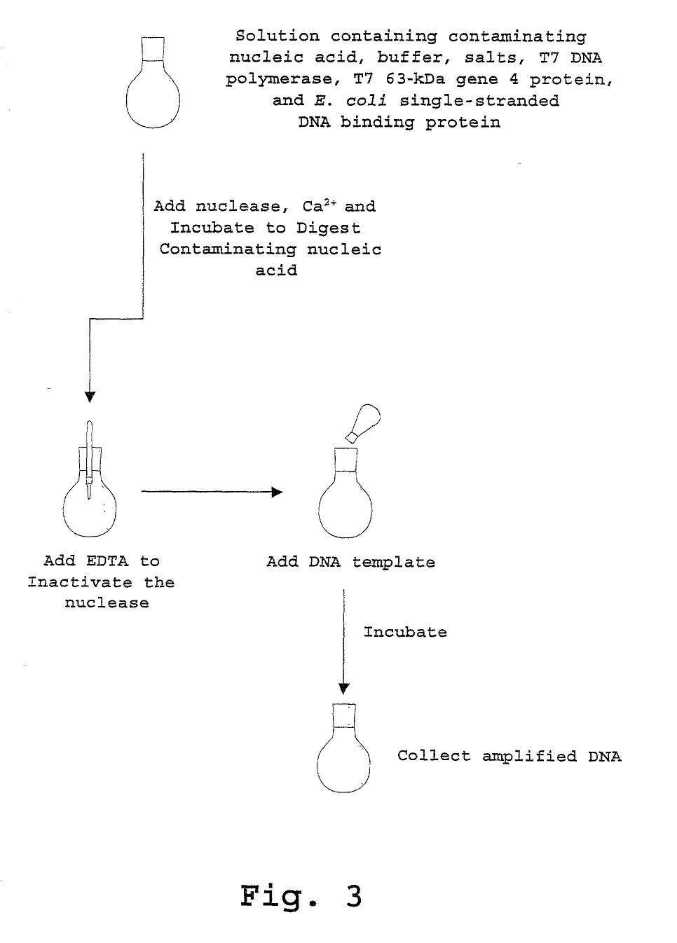

In accordance with certain preferred embodiments, and referring to FIG. 3, the method disclosed here may also be used to amplify the DNA under isothermal conditions. Suitable methods for isothermal amplification of DNA are disclosed in U.S. Pat. No. 4,946,786, and WO 00/41524, the entire disclosure of each of which is incorporated herein by reference for all purposes. Other isothermal DNA amplification methods are well known to those skilled in the art and, given the benefit of this disclosure, can be used in combination with the methods disclosed here. Typically, the reaction mixture for amplification of DNA under isothermal conditions comprises a suitable buffer, salts such as Mg²⁺, K⁺, etc, deoxyribonucleotides and ribonucleotides, a T7 DNA polymerase, or other similarly functioning polymerase, a T7 63-kDa gene 4 protein, or other T7 protein, and E. coli single-stranded DNA binding protein. In preferred embodiments, micrococcal nuclease and calcium ions are added to the solution to digest any unwanted contaminating nucleic acid. After incubation for a sufficient time, EDTA or EGTA may be added to inactivate the micrococcal nuclease. Subsequent to inactivation, the DNA template to be amplified may then be added to the solution, and no step of removing the inactivated micrococcal nuclease is necessary. The DNA template is amplified after a sufficient incubation time at a suitable temperature. Suitable temperatures and conditions for isothermal DNA amplification can be selected by skilled in the art given the benefit of this disclosure.

In accordance with certain preferred embodiments, the step of inactivating the micrococcal nuclease may be accomplished by displacing any bound calcium ions with a metal species having a higher affinity for the calcium ion sites, e.g. a lower dissociation constant K_{D} for the calcium ion sites. While not wishing to be bound by any particular scientific theory, it is believed that certain divalent and trivalent metal species bind to calcium ion sites with a higher affinity than calcium ions (Evans, C. H.). Therefore, addition of suitable amounts of divalent and trivalent metal species may provide for displacement of the bound calcium ions and subsequent inactivation of the enzyme, since most of the divalent and trivalent metals species do not permit functioning of the enzyme in the same way as bound calcium ions (potentially because of the higher valency of trivalent species such as lanthanides). When divalent and trivalent metal species are used it is preferable that suitable metal species are used which have no adverse effect on the functioning of the DNA polymerase. That is, the divalent and trivalent metal species should render then nuclease inactive without affecting the activity of any polymerases or other enzymes involved in the DNA synthesis reactions. In other embodiments, both EDTA or EGTA and divalent and trivalent metal species are used together to inactivate the enzyme. Such combination provides an additional measure to ensure that the nuclease is rendered inactive.

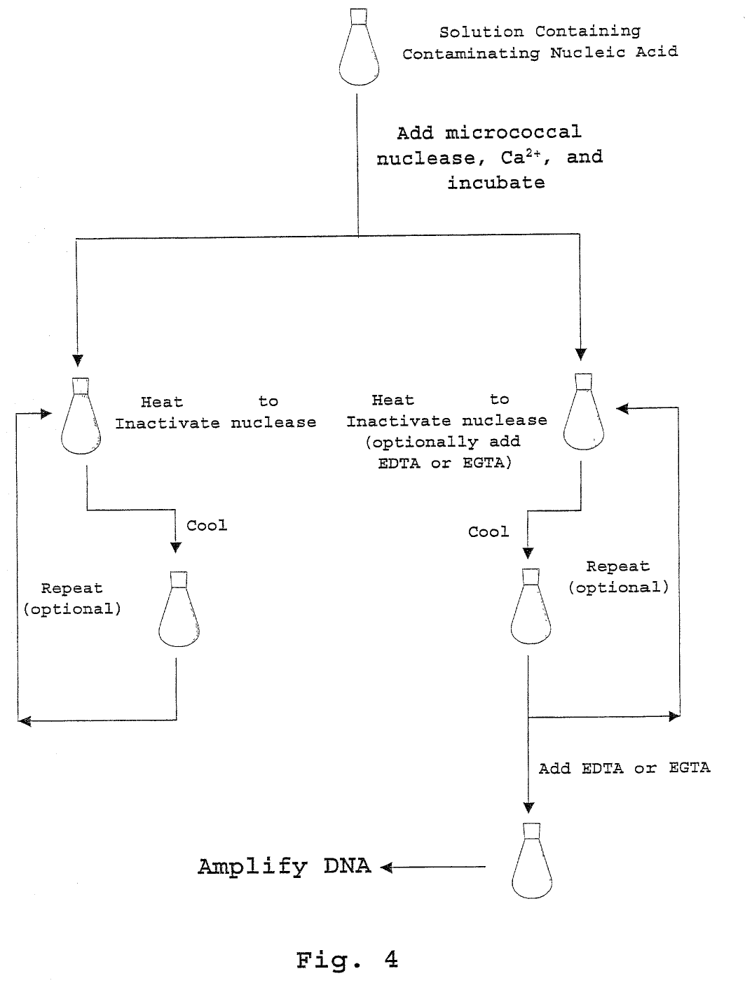

In accordance with certain preferred embodiments, the solution comprising the micrococcal nuclease can be heated to denature the enzyme. Such heating may be accomplished by increasing the temperature to a suitable temperature for denaturing the nuclease but allowing the DNA polymerase to remain active, e.g. 90° C. (see FIG. 4). Because the DNA polymerases are usually enzymes derived from thermophilic bacteria, and thus suitable for use in high temperatures assays such as PCR, preferably there is little or no adverse effect on the DNA polymerase from the heating process, whereas the micrococcal nuclease is denatured and its function is not restored when the temperature of the solution is lowered. The heating process may be repeated numerous times to ensure that the micrococcal nuclease is denatured. According to one embodiment, the solution comprising the micrococcal nuclease is thermally cycled several times prior to addition of any DNA template, primers or free nucleotides. This process is especially advantageous because a single thermal cycling apparatus may be used to accomplish the heating process to denature the nuclease and subsequent heating and cooling steps to amplify the DNA. In certain preferred embodiments, the heating process is used in combination with the addition of EGTA and/or trivalent and divalent metal species to ensure that the nuclease is inactive prior to addition of the DNA template.

In accordance with certain preferred embodiments, a kit is provided comprising enzyme, e.g. micrococcal nuclease, activator, e.g. calcium ions, and an inactivating agent such as EDTA or EGTA. The kit may be used for washing glassware, plastic tubes, and the like prior to performing DNA analysis. For example, prior to sequencing DNA, using mass spectroscopy or other suitable sequencing techniques, the apparatus, such as capillaries, injection syringes, etc., may be treated with active enzyme to digest any contaminating nucleic acid. In the case of mass spectroscopy, the digested free nucleic acids will not interfere with detection of the DNA fragments, since the fragments and the free nucleotides will have very distinct m/z ratios. The kit may also be used to ensure that no contaminating nucleic acid is present, such as in forensic applications, prior to performing sequencing of DNA in blood, urine, tissue, hair samples, and the like.

In accordance with certain preferred embodiments, a kit comprising reagents for performing polymerase chain reaction, the reagents comprising a DNA polymerase, primers, free nucleotides, and at least one buffer comprising magnesium ions, and a first enzyme capable of digesting contaminating nucleic acids to produce inert free nucleic acids, the first enzyme having maximal activity in the presence of bound activator and minimal activity in the absence of bound activator is provided. The kit is suitable for use with automated thermal cycling apparatus. Preferably, the free nucleotides include but are not limited to dATP, dGTP, dCTP and dTTP. The primers can be readily constructed by those skilled in the art given the benefit of this disclosure and the sequence of the primers typically depend on the sequence of the DNA template to be amplified. The DNA polymerase can be any of those commonly used in PCR reactions, such a Taq polymerase or Pfu polymerase, for example. Suitable buffers and salts will be readily selected by those skilled in the art given the benefit of this disclosure and typically depend on the DNA polymerase selected and the PCR conditions. Preferably, the enzyme capable of digesting contaminating nucleic acid comprises micrococcal nuclease. The activator preferably is calcium ions and EDTA or EGTA is typically added to remove the bound activator from the enzyme.

Although micrococcal nuclease has been identified as one selectively activatable and/or inactivatable enzyme useful in the present invention, one skilled in the art will readily be able to identify other useful selectively activatable and/or inactivatable enzymes. In addition, fragments and/or modifications of micrococcal nuclease which retain their ability to digest nucleic acids in a manner similar to the micrococcal nuclease itself are also useful in the present invention.

Several examples of the novel technology disclosed here are described below. The examples are not intended to limit the scope of this novel technology in any manner.

### EXAMPLE 1

### Use of Micrococcal Nuclease to Remove Contaminating DNA from a PCR Reaction Mixture

Micrococcal nuclease is added to remove contaminating DNA from a PCR reaction mixture. Micrococcal nuclease is obtained from Sigma (Sigma catalog number N5386. from Staphylococcus aureus, Foggi strain; 100-300 units/mg). EGTA, Ethylene glycol-bis(2-aminoethyl ether)-N,N,N′,N′-tetraacetic acid, is used as the micrococcal inactivating-agent and is obtained from Sigma (Sigma catalog number E8145. Tetrasodium salt). The reaction mixture (40 μl) contains 10 mM Tris-HCl, pH 8.3, 50 mM KCl, 3 mM MgCl₂, 0.5 mM CaCl₂, 5 units of AmpliTaq Gold™ (Applied Biosystems, Inc., Foster City, Calif.), and 1 μg (0.2 units) of micrococcal nuclease. The mixture is incubated at 37° C. for 60 min. 1 mM EGTA is then added to inactivate the micrococcal nuclease. Forward and reverse primers and the DNA template are added, and PCR is then carried out using standard conditions well known to those skilled in the art.

### EXAMPLE 2

### Removal of Contaminating DNA from an Isothermal DNA Synthesis Reaction Mixture

Micrococcal nuclease is used to remove contaminating DNA from an isothermal DNA synthesis reaction mixture. Micrococcal nuclease is obtained from Sigma (Sigma catalog number N5386 from Staphylococcus aureus, Foggi strain; 100-300 units/mg). EGTA, Ethylene glycol-bis(2-aminoethyl ether)-N,N,N′,N′-tetraacetic acid, is used as the micrococcal inactivating-agent and is obtained from Sigma (Sigma catalog number E8145. Tetrasodium salt). DNA synthesis in a T7 DNA replication system is so efficient that single molecules of DNA can be detected by amplification. Because synthesis is nonspecific, it is critical to remove all contaminating DNA from the mixture in order for synthesis to be specific for the added DNA. The reaction mixture (40 μL) contains 20 mM Tris-glutamate, pH 7.5, 15 mM MgCl₂, 1 mM CaCl₂, 5 mM dithiothreitol, 100 mM potassium glutamate, 10% DMSO, 7% Dextran T-500 polysaccharide (Amersham Pharmacia Biotech, Inc., Piscataway, N.J.), 500 μM each dGTP, dATP, dTTP and dCTP, and 300 μM rGTP, rATP, rCTP and rUTP, 1 μg of Δ28 T7 DNA polymerase, 1 μg of T7 63-kDa gene 4 protein, and 0.3 μg of E. coli single-stranded DNA binding protein, and 1 μg of micrococcal nuclease (Sigma Chemical Co.). Δ28 T7 DNA polymerase is a one-to-one mixture of E. coli thioredoxin and T7 gene 5 protein with a deletion of residues 118 to 145, as described in Tabor and Richardson, J. Biol. Chem. 264:6647, 1989, and U.S. Pat. Nos. 4,942,130 and 4,946,786, the entire disclosure of each of which is incorporated herein by reference. It is the same as Sequenase Version 2.0 enzyme sold by Amersham Pharmacia Biotech, Inc. (Piscataway, N.J.). T7 63-kDa gene 4 protein is the G4A_{G64} protein described in Mendelman et al., Proc. Natl. Acad. Sci. USA 89:10638, 1992 and Mendelman et al., J. Biol. Chem. 268:27208, 1993; it is the wild-type gene 4 63 kDa protein except that the methionine at residue 64 has been replaced with a glycine to prevent initiation of synthesis of the 56-kDa form of the gene 4 protein. E. coli single-stranded DNA binding protein (SSB) is overproduced and purified as described in Lohman et al., Biochemistry 25:21 (1986). It can also be purchased from Amersham Pharmacia Biotech, Inc., Piscataway, N.J. This mixture is incubated at 30° C. for 60 min. 2 mM EGTA is added to inactivate the micrococcal nuclease, and the DNA template to be amplified is then added. The reaction mixture is then incubated at 30°C. for 60 min.

DNA was quantitated by its ability to stimulate a real time DNA amplification reaction based on the T7 replication system, as described in PCT number WO 00/41524, published Jul. 20, 2000. In this assay, the time required for the synthesized DNA to reach a designated threshold fluorescence value is proportional to the log of the input concentration of DNA over more than a 10,000,000-fold range of DNA concentration. In a typical example, in which DNA synthesis is measured by the presence of the dye SYBR Green in the mixture, in the absence of added DNA the reaction requires 90 min. to reach 100 relative fluorescence units (RFU). In the presence of 100 picograms of pUC19 DNA, the time required to reach 100 RFU is reduced to 10 min. This value is the same regardless of whether micrococcal nuclease is present in the mixture, since it has been inactivated by the addition of EGTA. If the 100 picograms of pUC19 DNA are added to the reaction mixture during the incubation reaction when micrococcal nuclease is active (1 μg of nuclease in the presence of 1 mM CaCl₂, as described above), then the time required to reach 100 RFU is again 90 min. These data indicate that under these conditions much greater than 99.99% of the template DNA has been rendered inactive by the digestion with micrococcal nuclease, and that micrococcal nuclease has no effect on the presence of DNA after it has been inactivated by the addition of EGTA.

Each of the following citations is incorporated herein by reference in its entirety for all purposes.

1. Cunningham, L. J. Am. Chem. Soc. 80, 2546 (1958).

2. Privat de Garilhe, M., Cunningham, L., Laurila, U. R. and Laskowski, M. J. Biol. Chem. 224, 751 (1957).

3. Alexander, M., Heppel, L. A. and Hurwitz, J. J. Biol. Chem. 236, 3014 (1961).

4. Dingwall, C., Lomonossoff, G. P., and Laskey, R A. High sequence specificity of micrococcal nuclease. Nucleic Acids Res. 9(12):2659-73 (1981).

5. Sulkowski, E., Odlyzko, A. M. and Laskowski, M. Preparation of 3′-dinucleotides from homoribopolymers by digestion with micrococcal nuclease. Anal. Biochem. 38(2):393-400 (1970).

6. Sulkowski, E. and Laskowski, M. Action of micrococcal nuclease on polymers of deoxyadenylic and deoxythymidylic acids. J. Biol. Chem. 244(14):3818-22 (1969).

7. Telford, D. J. and Steward, B. W. Micrococcal nuclease: its specificity and use for chromatin analysis. Int. J. Biochem. 12(2):127-37 (1989)

8. Evans, C. H. Biochemistry of the Lanthanides. New York: Plenum Press (1983)

It is to be understood that the embodiments of the invention that have been described are merely exemplary and illustrative of some applications of the principles of the invention. Numerous additions, omissions, and modifications will be readily apparent to those skilled without departing from the true spirit and scope of the invention.

What is claimed is:

1. A method comprising:
rendering contaminating nucleic acid inert by:
adding an enzyme to a solution, the enzyme having maximal activity in the presence of bound activator and minimal activity in the absence of bound activator and the enzyme capable of rendering contaminating nucleic acid inert in the presence of bound activator and incapable of rendering contaminating nucleic acid inert in the absence of bound activator;
incubating enzyme having bound activator to allow the enzyme to render the contaminating nucleic acid inert; and
removing bound activator to inhibit the enzyme from further acting on nucleic acid.

2. The method of claim 1 in which the enzyme renders the contaminating nucleic acid inert by degrading the contaminating nucleic acid to yield phosphate moieties at the 3′ ends and hydroxyl moieties at the 5′ ends of nucleotides or nucleotide fragments.

3. The method of claim 1 in which the enzyme is micrococcal nuclease.

4. The method of claim 3 in which the bound activator is calcium ions.

5. The method of claim 4 in which the bound activator is removed by adding EDTA or EGTA.

6. The method of claim 1 further comprising adding a DNA template, a DNA polymerase, primers and optionally free nucleotides to the solution to amplify the DNA template.

7. The method of claim 6 in which the DNA template is amplified using polymerase chain reaction.

8. The method of claim 6 in which the DNA template is amplified using isothermal methods.

9. The method of claim 1 further comprising adding an effective amount of additional activator to allow the enzyme to bind to the additional activator and digest remaining contaminating nucleic acid.

10. The method of claim 9 further comprising removing the additional bound activator to inhibit the enzyme from further acting on nucleic acid.

11. A method of digesting contaminating nucleic acid comprising:
adding an enzyme to a solution for performing a DNA synthesis reaction, the enzyme capable of digesting the contaminating nucleic acid in a first state and incapable of digesting the contaminating nucleic acid in a second state;
converting the enzyme into the first state by adding an activator;
incubating the enzyme in the first state to allow for digestion of the contaminating nucleic acid by the enzyme in the first state; and
converting the enzyme to the second state by adding an inactivating agent.

12. The method of claim 11 in which the enzyme is a selectively activatable enzyme in the first state and a selectively inactivatable enzyme in the second state.

13. The method of claim 11 in which the enzyme comprises micrococcal nuclease.

14. The method of claim 13 in which the activator comprises calcium ions.

15. The method of claim 14 in which the inactivating agent comprises EDTA or EGTA.

16. The method of claim 11 in which the enzyme degrades the contaminating DNA to yield phosphate moieties at the 3′ ends and hydroxyl moieties at the 5′ ends.

17. The method of claim 11 further comprising adding a DNA template, primers, and free nucleotides to the solution after the enzyme is converted to the second state.

18. A method of amplifying DNA comprising:
(a) treating one or more reagent solutions with an enzyme in a manner to digest contaminating nucleic acids, wherein the reagent solutions comprise at least a DNA polymerase, a buffer, free nucleotides and one or more salt solutions;
(b) inactivating the enzyme;
(c) adding a DNA template and nucleotide primers to the solution;
(d) amplifying the DNA template by using a process comprising the steps of:
(i) optionally denaturing a double-stranded DNA template if present;
(ii) allowing the primers to anneal to single stranded DNA and allowing binding of the DNA polymerase to the annealed DNA/primer complex; and
(iii) elongating the DNA strands using the free nucleotides and the DNA polymerase; and
(e) repeating step (d) for a predetermined number of cycles to amplify the DNA template.

19. The method of claim 18 wherein the enzyme degrades the DNA to yield phosphate moieties at the 3′ ends and hydroxyl moieties at the 5′ ends.

20. The method of claim 18 wherein the enzyme is micrococcal nuclease.

21. The method of claim 20 wherein the activator is calcium ions.

22. The method of claim 21 wherein the inactivating agent comprises EDTA or EGTA.

23. An isothermal method of amplifying DNA comprising:
(a) treating one or more reagent solutions with an enzyme in a manner to digest contaminating nucleic acids, wherein the reagent solutions comprise at least a DNA polymerase, free deoxyribonucleotides, free ribonucleotides, a buffer, and one or more salt solutions;
(b) inactivating the enzyme;
(c) adding a DNA template to the solution;
(d) incubating the solution comprising the DNA template to amplify the DNA template.

24. The method of claim 23 in which the enzyme is inactivated by heating the reagent solution to a suitable temperature to denature the enzyme.

25. The method of claim 23 in which the enzyme is micrococcal nuclease.

26. The method of claim 25 in which the activator is calcium ions.

27. The method of claim 26 in which the inactivating agent comprises EDTA or EGTA.

28. The method of claim 23 in which the contaminating nucleic acid is DNA or RNA.

29. A kit comprising:
an enzyme capable of digesting contaminating nucleic acids in a manner to produce nucleotides having a 3′-phosphate group and a 5′-hydroxyl group;
an activator capable of binding to the enzyme and activating the enzyme to allow the enzyme to digest the contaminating nucleic acids to produce the nucleotides having a 3′-phosphate group and a 5′-hydroxyl group; and
an inactivating agent operative produce an inactive enzyme.

30. The kit of claim 29 wherein the enzyme comprises micrococcal nuclease.

31. The kit of claim 30 wherein the activator is calcium ions.

32. The kit of claim 31 wherein the inactivating agent is EDTA or EGTA.

33. A kit comprising:
reagents for performing polymerase chain reaction, the reagents comprising a DNA polymerase, primers, free nucleotides, and at least one buffer comprising magnesium ions; and
a first enzyme capable of digesting contaminating nucleic acids to produce inert free nucleic acids, the enzyme having maximal activity in the presence of bound activator and minimal activity in the absence of bound activator.

34. The kit of claim 33 in which the first enzyme comprises micrococcal nuclease.

35. The kit of claim 34 in which the bound activator is calcium ions.

36. The kit of claim 35 in which EDTA or EGTA removes the bound activator from the enzyme.

37. A method of digesting nucleic acids comprising the steps of:
adding a selectively activated enzyme to a solution comprising nucleic acids;
adding an activator to the solution comprising the selectively activated enzyme to activate the selectively activated enzyme;
incubating the solution to allow for digestion of the nucleic acids; and
inactivating the selectively activated enzyme.

38. A method of digesting contaminating nucleic acid in a DNA synthesis reaction comprising the steps of:
adding a selectively inactivatable enzyme to a solution comprising contaminating nucleic acid;
adding an activator to the solution comprising the selectively inactivatable enzyme to activate the selectively inactivatable enzyme;
incubating the solution to allow for digestion of the contaminating nucleic acid;
inactivating the selectively inactivatable enzyme; and
adding a DNA template to the solution for amplification of the DNA template.

39. A method of amplifying DNA comprising:
(a) treating one or more reagent solutions with an enzyme in a manner to digesting contaminating nucleic acids, wherein the reagent solutions comprise at least a DNA polymerase, a buffer, free nucleotides and one or more salt solutions;
(b) inactivating the enzyme;
(c) adding a DNA template and optionally nucleotide primers;
(d) thermally cycling the solution by using a process comprising the steps of:
(i) optionally denaturing double-stranded DNA template if present;
(ii) allowing the primers to anneal to DNA template and allowing binding of the polymerase to the annealed DNA/primer complex; and
(iii) elongating the DNA strands using the free nucleotides and the polymerase;
(e) repeating step (d) for a predetermined number of cycles to amplify the DNA template.

40. An isothermal method of amplifying DNA comprising:
(a) treating one or more reagent solutions with an enzyme in a manner to digest contaminating nucleic acids, wherein the reagent solutions comprise at least a DNA polymerase, free deoxyribonucleotides, a buffer, and one or more salt solutions;
(b) inactivating the enzyme;
(c) adding a DNA template to the solution;
(d) incubating the solution comprising the DNA template to amplify the DNA template.

41. A method of digesting nucleic acids comprising:
combining a selectively activatable enzyme with an activator to activate the selectively activatable enzyme and a solution comprising nucleic acids;
incubating the solution to allow for digestion of the nucleic acids; and
inactivating the selectively activatable enzyme.

42. A method of digesting nucleic acids comprising:
combining an activated enzyme with a solution comprising nucleic acids;
incubating the solution to allow for digestion of the nucleic acids; and inactivating the activated enzyme.

43. The method of claim 6 wherein the DNA template is amplified using Ø29 DNA polymerase and random hexamers.
